(19) United States
(12) Patent Application Publication
(10) Pub. No.: US 2024/0325288 A1
(43) Pub. Date: Oct. 3, 2024

(54) CLEANSING BALM

(71) Applicant: The Procter & Gamble Company, Cincinnati, OH (US)

(72) Inventors: SHAWN DAVID MCCONAUGHY, CINCINNATI, OH (US); Mahmoud Daffalla Eljack, Lebanon, OH (US); Tricia Jean Rist, Mason, OH (US); David Arthur Sturgis, Montgomery, OH (US); David Frederick Swaile, Cincinnati, OH (US)

(21) Appl. No.: 18/618,156

(22) Filed: Mar. 27, 2024

Related U.S. Application Data
(60) Provisional application No. 63/492,567, filed on Mar. 28, 2023.

(51) Int. Cl. A61K 8/92; A61K 8/34; A61K 8/37; A61K 8/60; A61K 8/73; A61Q 19/10

(52) U.S. Cl. CPC A61K 8/925; A61K 8/342; A61K 8/345; A61K 8/37; A61K 8/375; A61K 8/602; A61K 8/732; A61K 8/922; A61Q 19/10

(57) ABSTRACT

A stable, anhydrous, soft solid cleansing balm composition that includes greater than 50% of a natural or naturally derived emollient and wax structurant, and an emulsifier. The wax structurants can include one or more first ester waxes having a melting point of at least 55° C., one or more second ester waxes having a melting point of at least 45° C.; and optionally one or more fatty alcohols that are solid at 25° C.

### FIELD OF THE INVENTION

The present disclosure relates to a facial cleanser, in particular an oil-based cleansing balm that includes natural or naturally derived waxes as a structurant.

### BACKGROUND OF THE INVENTION

Many consumers want a facial cleanser that removes excess oil, impurities, makeup, sunscreen, and other skincare products while gently cleansing the skin leaving skin feeling hydrated and silky smooth.

One product form that can deliver these benefits is a cleansing balm. A cleansing balm is generally an oil-based product form that can be used alone or in combination with traditional liquid, micellar, or bar facial cleansers to help remove dirt and oil without overly drying the skin. Cleansing balms are generally sold in a tub-like jar, and it is ideally a creamy homogenous soft solid at room temperature. A consumer either uses an implement or their hand to scoop out a portion of balm and then massages it into their skin, which causes the creamy balm texture to melt into an oil. Then, the user adds warm water, emulsifying the oils and rinsing away skin care products, impurities, and excess oil, leaving the consumer with clean, hydrated skin.

To achieve the unique consistency, cleansing balms generally include a structurant. The structurant helps to provide the creamy, soft solid hardness and texture and maintains physical stability throughout the lifetime of the product, which generally includes shipping, handling, and storage at conditions that are not climate controlled. Current products can use polymer structurants that can include polyethylene, petroleum derived ingredients (e.g., paraffin, isoparaffin, petrolatum), and/or polyethylene glycol (PEG) ingredients.

However, some consumers prefer products with a natural or naturally derived structurant. Many natural waxes are not ideal or compatible for formulating with the high level of oil to create a cleansing balm that has a consumer acceptable feel, is stable at high and low temperatures, and has appropriate hardness throughout the composition.

Therefore, there is a need for a phase stable, homogeneous, oil-based soft solid cleansing balm that contains one or more natural or naturally derived structurants.

### SUMMARY OF THE INVENTION

A soft solid cleansing balm composition comprising: (a) greater than 50% of an emollient comprising a melting point below 40° C.; wherein the emollient is natural or naturally derived; (b) an emulsifier; (c) from about 8% to about 28% of a combination of wax structurants comprising: (i) one or more first ester waxes comprising a melting point of at least 55° C., preferably at least about 60° C.; (ii) one or more second ester waxes comprising a melting point of at least 45° C.; and (iii) optionally, from about 0.2% to about 3% of one or more fatty alcohols; wherein the fatty alcohols are solid at 25° C.; wherein the cleansing balm is anhydrous; and wherein the cleansing balm composition is formulated without synthetic waxes.

A soft solid cleansing balm composition comprising: (a) a natural or naturally derived emollient; wherein the emollient is liquid at 25° C.; (b) an emulsifier; (c) a naturally-derived wax blend comprising at least two ester waxes comprising a melting point of at least 45° C. and optionally, one or more fatty alcohols; wherein the fatty alcohols are solid at 25° C.;

wherein the cleansing balm is anhydrous; wherein the cleansing balm composition is formulated without polyethylene; wherein the composition comprises an average hardness of greater than 50 gf and less than 350 gf; wherein the cleansing balm comprises a skin to bulk hardness ratio greater than 0.60 and less than 2.0; and wherein the composition comprises an oil binding capacity percent of at least 65%.

A cleansing balm composition comprising: (a) a natural or naturally derived emollient; wherein the emollient is liquid at 25° C.; (b) a non-ionic emulsifier; (c) a naturally-derived wax blend comprising: (i) a C18-36 triglyceride; (ii) tribehenin; (iii) one or more ester waxes comprising jojoba esters, trilaurin, shea butter, beeswax, polyglyceryl-3 beeswax, or a combination thereof; wherein the cleansing balm is anhydrous; wherein the cleansing balm composition is formulated without polyethylene and silicone oils.

### DETAILED DESCRIPTION OF THE INVENTION

An anhydrous, oil-based cleansing balm can efficiently remove makeup including waterproof mascara, sunscreen, and other skincare products and impurities. However, if these products are formulated incorrectly, they can leave a user's skin feeling greasy and grimy instead of clean, hydrated, and silky smooth. In addition, the cleansing balms need to have an appropriate hardness and texture, so the consumer can easily scoop the composition from the jar without it feeling mushy or runny. The cleansing balm cannot phase separate throughout the shelf life of the product, which includes shipping, handling, and prolonged storage in facilities that are not climate controlled.

A suitable concentration of structurant, such as a wax structurant, can help provide the compositions with the desired product performance, texture, hardness, stability, and/or to otherwise help suspend any dispersed solids or liquids within the composition. Traditionally, cleansing balms used synthetic or petroleum-based waxes including polyethylene as the structurant. However, many consumers prefer skin care products formulated without petroleum-derived ingredients and include structurants that are natural or naturally derived, and it is a significant challenge to find natural or naturally derived waxes that produce an appropriate hardness with a crystal structure throughout the balm.

For example, there are minimal interactions in a composition with polyethylene wax and triglyceride and ester oils. This is because the wax and oils all have straight carbon chains without any complex bends caused by double bonds or additional functional groups like esters, which limits intermolecular forces. Potential interactions between an emollient and a natural or natural derived wax can be much more complicated.

In some examples, it can be preferred to have a natural or naturally derived emollient and structurant. However, the more natural or naturally derived the emollient and wax become, often the more complicated the interactions between the two and different molecular rearrangements can happen during crystallization. Such rearrangements result in differing crystalline material properties, such as hardness, viscoelasticity, and oil encapsulation efficiency. Inter-molecular forces, such as van der Waals forces, hydrogen bonds, and London dispersion forces, can play a role in co-crystallization of waxes.

Specifically, with ester-based waxes, this interaction and polymorphism can create cleansing balms that appear to be consumer acceptable on the top of the balm but are unacceptable to the consumer because they are significantly softer and mushier than appearance would suggest. These same compositions may have lower oil absorption and after the consumer has used the product once, creating a large divot in the product, the divot will fill with oil. Conversely some naturally derived emollient and structurant combinations may result in cleansing balms with suitable visual appearance but they are too hard. Cleansing balms that are too hard require extra force from the consumer to scoop from the jar. This results in a fracture plane where oil visibly separates from the composition and results in undesirable chunks of the product being spread on the skin versus a soft smooth composition.

A cleansing balm where natural structurants crystalize in the presence natural emollients and create both a preferred hardness and oil binding capacity is challenging to create and difficult to predict due to the diverse and complex chemical structures of naturally derived ingredients. It was found that a cleansing balm with about 6% to 17% of an ester wax having a melting point of at least about 55 C; and from about 3% to 17% of an ester wax having a melting point of at least about 45 C; and from about 0.01% to 1.5% of a fatty alcohol or fatty alcohol-containing wax creates a consumer preferred and stable cleansing balm.

The cleansing balm can be physically stable. A composition is physically stable if by visual detection there is no phase separation, including weeping of oil onto the surface, and the composition has a % oil binding capacity of greater than 65%, according to the Oil Binding Capacity Test Method, described herein. As used herein, “visual detection” means that a human viewer can visually discern if the product has separated into 2 distinct layers in a 2 oz clear glass jar with the unaided eye (excepting standard corrective lenses adapted to compensate for near-sightedness, farsightedness, or stigmatism, or other corrected vision) in lighting at least equal to the illumination of a standard 100-watt incandescent white light bulb at 30 cm. Weeping of oil can at times be observed directly as droplets of liquid on the surface of the product, but may also be observed as weeping of oil into an intentionally created divot on the surface of the product that is approximately 1 mm deep and 3 mm long. If the composition appears stable upon visual detection, then the composition can also have a % oil binding capacity of greater than 65%, according to the Oil Binding Capacity Test Method, described herein, to be considered physically stable.

In some examples, the composition can be formulated without, substantially free of, or free of silicone oils. In some examples, the composition can be formulated without, substantially free of, or free from certain waxes, such as, for example, polyethylene and other petroleum derived waxes, synthetic wax, paraffins, isoparaffins, ozokerite. In some examples, the composition can be formulated without, substantially free of, or free of water.

All percentages are by weight of the cosmetic composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 21° C. and at ambient conditions, where “ambient conditions” means conditions under about 1 atmosphere of pressure and at about 50% relative humidity. All numeric ranges are inclusive of narrower ranges; delineated upper and lower range limits are interchangeable to create further ranges not explicitly delineated.

### Definitions

The term “anhydrous” as used herein means substantially free of added or free water. From a formulation standpoint, this means that the anhydrous cleansing balm compositions of the present invention contain less than about 2%, and more specifically less than 1%, more specifically less than 0.25%, and even more specifically zero percent by weight of free or added water, other than the water of hydration typically associated with ingredients in the cleansing balm and water brough in by the inclusion of natural extracts.

As used herein, “clean” refers to cosmetic compositions that are formulated without the following ingredients: Acrylate Monomers, Aluminum Salts, Animal Musk's/Fats/Oils, Benzophenones and related compounds, Butoxyethanol, Butylated hydroxyanisole (BHA), Butylated hydroxytoluene (BHT), Carbon Black or Black 2, Coal Tar, Ethanolamines, Formaldehyde and Formaldehyde-releasing agents, Hydroquinone, Lead and Lead Acetate, Mercury and Mercury Compounds (Thimerisol), Methoxyethanol, Methyl Cellosolve, Methylchloroisothiazolinone and Methylisothiazolinone, Mineral Oil, Nanomaterials, Chemical sunscreens (including oxybenzone, avobenzone, and octinoxate), Parabens (including butyl-, methyl-, and propylparaben), Petrolatum and Paraffin, Phenoxyethanol, Phthalates, Plastic Microbeads, Polyethylene, Synthetic wax, paraffin wax, ozokerite, Polyacrylamide and Acrylamide, Styrene, Sulfates (including sodium lauryl sulfate and sodium laureth sulfate), Talc, Toluene, Triclosan and Triclocarban, Acetaldehyde, Acetone, Acetonitrile, Benzalkonium chloride, Bisphenol A (BPA), Ethylenediaminetetraacetic acid (EDTA), Methylene chloride, Polytetrafluoroethylene (PTFE), Perfluorooctanoic acid (PFOA), 1,4 Dioxane, Octinoxate, Nitromusks and Polycyclic Musks, PFAS compounds, Ethoxylated Ingredients, Polyethylene glycol (PEG), Cyclic Silicones, volatile linear silicones including silicone elastomers which contain volatile linear silicones, and derivatives and combinations thereof.

As used herein, “formulated without” means that the ingredient is not intentionally added. However, “formulated without” does not guarantee “100% free from” since trace contaminants are possible.

“Improve the appearance of” means providing a measurable, desirable change or benefit in skin appearance, which may be quantified, for example, by a decrease in redness, inflammation, and/or plaque scales.

As used herein, “natural” refers to cosmetic ingredients obtained only from plants, animals, micro-organisms, or minerals according to International Organization for Standardization 16128-1, Part 1, ¶2.1 (Feb. 15, 2016). Ingredients obtained from fossil fuels are excluded from the definition. A non-limiting list of “natural” ingredient sources can include olive, palm, corn, palm, rapeseed, soybean, sunflower, coconut, tallow, canola, castor, and combinations hereof.

As used herein, “naturally derived” refers to cosmetic ingredients of greater than 50% natural origin by molecular weight, by renewable carbon content or by any other relevant methods, obtained through defined chemical and/or biological processes with the intention of chemical modification according to ISO 16128-1, Part 1, ¶3.1 (Feb. 15, 2016). ISO 16128-1, Appendix B includes a non-limiting list of chemical and biological processes for derived natural ingredients, which is incorporated by reference.

As used herein, “skin care active” means a compound or combination of compounds that, when applied to skin, provide an acute and/or chronic benefit to skin or a type of cell commonly found therein. Skin care actives may regulate and/or improve skin or its associated cells (e.g., improve skin elasticity, hydration, skin barrier function, and/or cell metabolism).

As used herein, “structurant” means any material known or otherwise effective in providing suspending, gelling, viscosifying, solidifying, and/or thickening properties to the composition or which otherwise provide structure to the final product form.

As used herein, “substantially free” means less than 0.5%, less than 0.25%, less than 0.1%, less than 0.05%, and/or less than 0.01%.

### Cleansing Balm Composition

The composition can be a cleansing balm with a hardness of a soft solid, which is readily deformed upon application of force by a consumer when scooping out of the package and spread on the face. When stored at a jar at room temperature, if the jar is tipped the composition will not visibly move. It can be important for the cleansing balm to have an appropriate average hardness, not being too soft and mushy, but also not being too hard for a consumer to easily scoop and apply the composition.

In addition, it can be important for the cleansing balm to have a consistent hardness between the surface and middle of the product. A balm with a very rigid surface but a mushy center is undesirable, conversely a balm with a very hard skin that is difficult to scoop is also undesirable. However, a surface “skin” that is of similar hardness and may even be slightly more rigid than the bulk of the product may be acceptable and even preferred. A measure of the skin to bulk hardness is described in the hardness method, the skin to bulk hardness ratio can be from about 0.60 to about 2.0, alternatively from about 0.80 to about 1.5, and alternatively from about 0.80 to about 1.25, as measured according to the Penetration Method described herein. Alternatively, the measure of the skin to bulk hardness ratio can be greater than 1.0, alternatively greater than 1.1, and alternatively greater than 1.2, as measured according to the Penetration Method described herein.

The composition can have an average hardness of greater than 50 gf and less than 350 gf, alternatively from about 50 gf to about 300 gf, alternatively from about 50 gf to about 250 gf, and alternatively from about 50 gf to about 200 gf as measured according to the Penetration Method described herein.

The composition can also be physically stable. The Oil Binding Capacity Test Method can help predict how much oil a cleansing balm can hold without weeping. To be physically stable, the % oil binding capacity can be greater than 65%, according to the Oil Binding Capacity Test Method. In another example, the % oil binding capacity can be greater than 70%, alternatively greater than 75%, alternatively greater than 80%, as measured according to the Oil Binding Capacity Test Method.

The composition may include a combination of one or more of the following: structurants, emollients, emulsifiers, skin actives, or combinations thereof. The structurants may include natural and naturally derived waxes that can be solid at 21° C. The emollients can be liquid at room temperature, these may include one or more of the following: alkanes, esters and triglycerides, or a combination thereof. The emulsifiers may be nonionic, anionic and/or cationic emulsifiers. In some examples, the emulsifiers can be non-ionic emulsifiers including ethoxylated and non-ethoxylated emulsifiers may be used.

### Structurants

The cleansing balm composition can include a suitable concentration of natural or naturally derived structurants to help provide the compositions with the desired texture, product hardness, and to help suspend any dispersed solids or liquids including the oils within the composition.

The cleansing balm can contain from about 5% to about 35%, alternatively from about 8% to about 28%, alternatively from about 10% to about 21%, alternatively from about 11% to about 17% of one or more wax structurants.

The compositions can include a combination of wax structurants. The composition can include one or more ester waxes having a melting point of at least 55° C., alternatively at least 60° C. In some examples the one or more ester waxes can have a melting point between 55-70° C. Non-limiting examples can include stearyl behenate, tribehenin, C18-36 acid triglycerides, hydrogenated rapeseed oil, and combinations thereof. The composition can include from about 4% to about 25%, alternatively from about 5% to about 18%, alternatively from about 8% to about 15%, and alternatively from about 10% to about 13%, by weight of the composition, of the one or more ester waxes having a melting point of at least 55° C.

The composition can include one or more ester waxes having a melting point between 45-55° C. Non-limiting examples can include jojoba esters, trilaurin, shea butter, beeswax, polyglyceryl-3 beeswax, and combinations thereof. The composition can include from about 1% to about 12%, alternatively from about 2% to about 10%, and alternatively from about 2% to about 6%, by weight of the composition, of the one or more ester waxes having a melting point between 45-55° C.

In some examples, the composition can include one or more wax structurants having a melting point higher than the ester waxes in the composition, specifically a melting point of at least 70° C., alternatively at least 80° C. Non-limiting examples can include Oryza sativa (Rice) bran wax. The composition can include from about 0.1% to about 2%, alternatively from about 0.2% to about 1%, by weight of the composition, of a wax structurant having a melting point of at least 75° C.

Some examples include at least one or more ester waxes having a melting point of at least 55° C. and at least one wax structurant having a melting point between 45-55° C. and/or at least one wax structurant having a melting point of at least 75° C. In these instances, the one or more ester waxes having a melting point of at least 55° C. make up greater than 50% of the total wax structurants, alternatively from about 55% to about 98%, alternatively from about 65% to about 97%, alternatively from about 68% to about 96%, and alternatively from about 70% to about 85%. Additional wax structurants may be added to modify the structure of the cleansing balm that can include fatty alcohol or fatty alcohol-containing waxes that are solids at 25° C. The composition can include from about 0.2% to about 3%, alternatively from about 0.3% to about 2%, alternatively from about 0.5% to about 1.5%, by weight of the composition, of a fatty alcohol.

Fatty alcohols can include monohydric alcohols having 8-22 carbon atoms, although longer chain alcohols in excess of 30 carbons may be used. The fatty alcohols may be saturated or unsaturated. The fatty alcohols may be straight or branched. In one example, the fatty alcohol may comprise straight chain, saturated fatty alcohol with a terminal hydroxyl. The fatty alcohols can include decyl alcohol, lauryl alcohol, myristyl alcohol, cetyl alcohol, stearyl alcohol, isostearyl alcohol, icosyl alcohol, behenyl alcohol, batyl alcohol, arachidyl alcohol, brassica alcohol, coconut alcohol, hydrogenated jojoba alcohol, jojoba alcohol, or a combination thereof. In some examples, the fatty alcohol can have an average carbon chain length from about 12 to about 22, alternatively from about 16 to about 22, alternatively from about 16 to about 18. Alternatively, the fatty alcohol can have an average chain length of from about 12 to about 18. The fatty alcohols can include cetyl and/or behenyl alcohol.

In some examples, the cleansing balm composition can be formulated without, substantially free of, or free of cetearyl alcohol because they believe that it can have unfavorable comedogenicity. In another example, the cleansing balm compositions can be formulated without, substantially free of, or free of palmitic acid and stearic acid.

### Emulsifiers

The cleansing balm composition can contain one or more emulsifiers. The emulsifiers useful in the present invention typically are oil soluble or miscible with the oil soluble external phase materials, especially at the temperature at which the lipid material melts. It also should have a relatively low HLB value. Emulsifiers suitable for use in the present invention have HLB values typically in the range of from about 1 to about 7 and can include mixtures of different emulsifiers. Preferably, these emulsifiers will have HLB values from about 1.5 to about 6, and more preferably from about 2 to about 5. The emulsifier can be substantially free or free of ethylene oxide repeat units.

The composition can contain from about 1% to about 30%, alternatively from about 5% to about 25%, alternatively from about 8% to about 23%, alternatively from about 13% to about 21%, and alternatively from about 14% to about 20% of an emulsifier. The composition can contain from about 1% to about 15%, alternatively from about 5% to about 13%, alternatively from about 7% to about 10%, of an emulsifier. The composition can contain less than 15%, alternatively less than 13%, alternatively less than 10%, alternatively less than 9%, and alternatively less than 5% of an emulsifier.

The emulsifier can include Sorbeth-30 Tetraoleate (Barcleanse STO, Barnet), Sorbitan Sesquioleate (Span 83, Croda), Glyceryl Laurate (ultrapure GML), Polyglyceryl-6 Dicaprate (Sunsoft Q-102H-C, Iwase Cosfa), Polyglyceryl-6 Distearate, Polyglyceryl-6 Myristate (Sunsoft Q-14Y-C, Iwase COSFA), Glyceryl Citrate/Lactate/Linoleate, Glycryl Caprylates, Polyglyceryl-4 Cocoate, Polyglyceryl-3 Caprate (Imwitor Lite Muls, EES Cosmetic Solutions), Polyglyceryl-6 Oleate, Potassium Olivate (Olcamuls low, DKSH), Peg-20 Glyceryl Triisostearate, PEG-10 Isostearate or a combination thereof.

Additional examples of emulsifiers can include sorbitan esters such as sorbitan laureates (e.g., SPAN® 20), sorbitan palmitates (e.g., SPAN® 40), sorbitan stearates (e.g., SPAN® 60 and SPAN® 65), sorbitan monoolcates (e.g., SPAN® 80), sorbitan triolcates (e.g., SPAN® 85), sorbitan sesquioleates (e.g., EMSORB® 2502), and sorbitan isostearates (e.g., CRILL® 6); polyglycerol esters and ethers (e.g., TRIODAN® 20); polyoxyethylene fatty acids, esters and ethers such as polyoxyethylene (2) oleyl ethers, polyethoxylated oleyl alcohols (e.g. BRIJ® 92 and SIMUSOL®92), etc.; mono-, di-, and triphosphoric esters such as mono-, di-, and triphosphoric esters of oleic acid (e.g., HOSTAPHAT KO3OON), polyoxyethylene sorbitol esters such as polyoxyethylene sorbitol hexastearates (e.g., TLAS® G-1050), ethylene glycol fatty acid esters, glycerol mono-isostearates (e.g., IMWITOR 780K), ethers of glycerol and fatty alcohols (e.g., CREMOPHOR WO/A), esters of polyalcohols, synthetic primary alcohol ethylene oxide condensates (e.g., SYNPERONIC A2), mono and diglycerides of fatty acids (e.g., ATMOS® 300), and the like. Other preferred emulsifiers include the diglycerol esters derived from monooleate, monomyristate, monopalmitate, and monoisostearate acids.

### Emollients

As consumers seek more natural ingredients in their skin care products, including cleansing balms, one approach to formulation is to use emollients derived from natural oils. Emollients derived from natural oils can be derived from plant sources, such as palm oil or coconut oil, and can be liquid at 25° C. One example of an emollient derived from natural oils may be a liquid triglyceride, such as caprylic/capric triglyceride, which is a triglyceride that is mild on the skin making it ideal for high use levels. Due to the requirement of cleansing balms to remove water resistant make-up additional emollient that are alkanes or esters may be needed to aid in solubilizing the makeup, including esters such as cetyl ethylhexanoate, and coco-caprylate/caprate. The cleansing balm composition can be formulated without, free of, or substantially free of petroleum-based emollients. The emollient can be a liquid emollient having a melting point below 40° C., below 35° C., or below 30° C. The liquid emollient can be an oil, which can include an ester, alkane, triglyceride, non-volatile silicones, and combinations thereof. In some examples, the cleansing balm composition can contain one or more liquid emollients.

The cleansing balm composition can include one or more of the following liquid emollients:
An alkane or isoalkane, ranging in total carbon from about C8-C60, including but not limited to squalane, squalene, coconut alkanes, coconut/palm kernel alkanes, C9-12 Alkane, C10-13 Alkane, C13-15 Alkane, C15-19 Alkane, C14-22 Alkane, C30-34 Alkane, C9-20 Isoalkane, C10-12 Isoalkane, C12-15 Isoalkane, isohexadecene, C32-54 Isoalkane, or a combination thereof.
An ester formed from an alcohol having from 1-6 carbons and acid having from about 6-22 carbons, including but not limited to isopropyl isostearate, isopropyl palmitate, isoamyl laurate, shea butter ethyl esters, shea butter cetyl esters, shea butter decyl esters, shea butter oleyl esters, jojoba ester oils, coco-capylate/caprate, ethyl caprate, ethylhexyl caprate, isoamyl caprylate/caprate, cetyl ethylhexanoate, argan oil isostearyl esters, babassu seed oil ethyl esters, camelina sative seed oil ethyl esters, castor/olive oil esters, coconut oil decyl esters, coconut oil ethyl esters, linseed oil ethyl esters, olive oil decyl esters, olive oil ethylhexyl esters, palm kernel oil triacetin esters, palmitoyl serine/silk amino acid methyl esters, rapeseed oil decyl esters, safflower seed oil decyl esters, sunflower seed oil cetyl esters, sunflower seed oil ethyl esters, trehalose isostearate esters, glyceryl caprate, or a combination thereof.
A triglyceride having 3 carbon chains where the carbon chain length ranges from about C7-C18, including but not limited to triheptanoin, caprylic/capric triglycride, C10-19 triglycerides, caprylic/capric/lauric triglycerides, hydrogenated C12-C18 triglycerides, triethylhexanoin, or a combination thereof.
A glycol having 2 or more alcohol groups, including but not limited to pentylene glycol, propylene glycol, dipropylene glycol, butylene glycol.
A non-volatile silicone fluid including but not limited to dimethicone, amodimethicone, or a combination thereof.

A relatively high level of oil may be necessary to sufficiently remove makeup including waterproof mascara, sunscreen and other skin care products, and impurities from the skin. The cleansing balm composition can include from about 40% to about 80%, alternatively from about 50% to about 75%, alternatively from about 60% to about 75%, alternatively from about 65% to about 75%, by weight of the composition of one or more emollients. The cleansing balm can include greater than 50%, greater than 55%, greater than 60%. Greater than 63%, greater than 65%, greater than and 70% emollient.

### Preservative

In some examples, the composition can be free of or substantially free of a traditional cosmetic preservative. Due to the anhydrous nature of the cleansing balm, the composition may not require a traditional preservative ingredient to inhibit microbial growth.

In other examples, the composition can include a traditional preservative such as phenoxyethanol.

Alternatively multi-functional ingredients, like phenylpropanol, may be primarily incorporated into the composition to provide both a functional benefit (e.g., fragrance, solvent) and can contribute to microbial hostility.

### Other Optional Ingredients

The present composition may optionally include one or more additional ingredients commonly used in cosmetic compositions (e.g., dyes, pigments, mica, insoluble particles, skin care actives, anti-inflammatory agents, sunscreen agents, emulsifiers, buffers, rheology modifiers, chelants, combinations of these and the like), provided that the additional ingredients do not undesirably alter the skin health or appearance benefits provided by the present compositions. The additional ingredients can be in the continuous phase and/or the dispersed phase. The additional ingredients, when incorporated into the composition, should be suitable for use in contact with human skin tissue without undue toxicity, incompatibility, instability, allergic response, and the like. Some nonlimiting examples of additional actives include vitamins, minerals, peptides and peptide derivatives, sugar amines, sunscreens, oil control agents, particulates, flavonoid compounds, hair growth regulators, anti-oxidants and/or anti-oxidant precursors, preservatives, protease inhibitors, tyrosinase inhibitors, anti-inflammatory agents, moisturizing agents, exfoliating agents, skin lightening agents, sunless tanning agents, lubricants, anti-acne actives, anti-cellulite actives, chelating agents, anti-wrinkle actives, anti-atrophy actives, phytosterols and/or plant hormones, N-acyl amino acid compounds, antimicrobials, and antifungals. In some examples oil soluble skin actives including retinoids such as retinol, retinyl propionate, retinyl palmitate may be included and deposited on the skin. In some examples oil soluble antioxidants including vitamin E derivatives such as tocopherol-acetate are included in the composition and deposited on skin. In some examples the composition may include vitamin C derivatives such as sodium ascorbyl phosphate, magnesium ascorbyl phosphate, ascorbyl palmitate, L-ascorbic acid, sodium ascorbate. In some examples, the composition can include extracts and oil based extracts, examples include; Curcuma Longa Root Extract, Carica Papaya Fruit Extract, Zingiber Officinale Root Oil, Coccinia Indica Fruit Extract, Solanum Melongena Fruit Extract, Melia Azadirachta Flower Extract, Citrus Aurantifolia Oil, Citrus Aurantium Dulcis Peel Oil, Amber Powder Cananga Odorata Flower Oil, Melia Azadirachta Leaf Extract, Moringa Oleifera Seed Oil, Echinacea Purpurea Root Extract, Helianthus Annuus Seed Oil, Ocimum Sanctum Leaf Extract, Citrus Aurantium Bergamia Fruit Oil, Moringa Pterygosperma Seed Extract, Corallina Officinalis Extract. In some examples the composition can include dyes including but not limited to green 3, green 5, yellow 5, yellow 10. In some examples, the composition can include a fragrance, in particular a natural fragrance or essential oil, or a colorant, in particular a natural colorant. In some examples, the cleansing balm composition can include a silicone elastomer including but not limited to silicone elastomers supplied solvated in non-volatile linear silicone fluids, silicone elastomers supplied solvated in alkanes, esters or triglycerides. The chelant can be selected from ethylenediaminetetraacetic acid (EDTA), trisodium ethylenediamine disuccinate (EDDS), trisodium dicarboxymethyl alaninate, tetrasodium glutamate diacetate, phytic acid, or a combination thereof. Other non-limiting examples of additional ingredients and/or skin care actives that may be suitable for use herein are described in U.S. Publication Nos. 2002/0022040; 2003/0049212; 2004/0175347; 2006/0275237; 2007/0196344; 2008/0181956; 2008/0206373; 2010/00092408; 2008/0206373; 2010/0239510; 2010/0189669; 2010/0272667; 2011/0262025; 2011/0097286; US2012/0197016; 2012/0128683; 2012/0148515; 2012/0156146; and 2013/0022557; and U.S. Pat. Nos. 5,939,082; 5,872,112; 6,492,326; 6,696,049; 6,524,598; 5,972,359; and 6,174,533.

When including optional ingredients in the compositions herein, it may be desirable to select ingredients that do not form complexes or otherwise undesirably interact with other ingredients in the composition, especially pH sensitive ingredients like niacinamide, salicylates and peptides. When present, the optional ingredients may be included at amounts of from 0.0001% to 50%; from 0.001% to 20%; or even from 0.01% to 10% (e.g., 50%, 40%, 30%, 20%, 10%, 5%, 4%, 3%, 2%, 1%, 0.5% or 0.1%), by weight of the composition.

In some examples, the composition can be formulated without, substantially free of, or free of dyes.

### Method of Use

The cleansing balm composition can be used as follows:
First a user, scoop a generous amount of the cleansing balm out of the package. They can use an implement, such as a spatula that can be included with the product upon purchase, or with their fingers. Then the consumer will warm the product in the palm of their hands and apply to dry skin. The product can be applied to any portion of the skin including the face, neck, and/or a portion or combination thereof.
Next, a consumer user's their fingertips to massage into skin, preferably with gentle upward and outward circular motions, and they can focus on areas that need attention (like eye makeup).
Then, the cleansing balm is rinsed with warm water. The user can use a warm, damp washcloth, makeup remover cloth, or other implement to help breakdown and remove makeup and other skin care compositions that a user may no longer want on their skin.
Optionally, the user can wash with their everyday facial cleanser.
The process can be repeated once or twice daily (AM/PM).

### Method of Making

All waxes, emollients and emulsifiers are combined in a vessel and heated, while stirring, to above the melting point of the highest melting point wax (typically heating to at least 75° C., some compositions may need heating to about 90° C.). Once all of the components are melted the composition is cooled, while stirring, to about 5° C. above the Krafft point (point at which one of the waxes begins to crystalize) of the system. The composition is poured into respective packaging, often a tub-like jar, and further cooled to transition from a molten liquid to a soft solid cleansing balm. Cooling in the jar can be accelerated by placing jars through a cooling tunnel, blowing cold air across the surface of the product, or placing the jars in controlled temperature rooms.

In some instances, it may be advantageous to blow cold air across the surface of the jar to accelerate formation of a solid layer, thereby preventing sloshing of the liquid out of the jar as the product proceeds down the manufacturing line and the jar is labeled. In other instances, it may be beneficial to blow hot air over the surface of the product to melt a layer of the product up to several millimeters in depth to create a smooth uniform surface prior to placing the cap on the jar.

### Test Methods

### Penetration Test to Determine Hardness

The penetration test is a physical test method that provides a measure of the firmness of waxy solids and soft solids and extremely thick creams and pastes. All hardness data reported herein were collected using a TA-XT2i Texture Analyzer with Texture Expert Exceed software (version 2.64). Hardness is measured in grams of force (gf).

Hardness is determined using a penetration method wherein a ¼ inch diameter ball probe (TA-8 as purchased from Texture Technologies Corp.) penetrates through a product depth of 10 mm at a rate of 1 mm/s. The trigger force to start data collection is set at 0.10 grams. The overall depth of product within the jar is >15 mm and no larger than a jar holding 100 ml of the molten composition with reasonable headspace. Multiple penetration measurements (minimum of 2 repetitions, preferably at least 3 repetitions) were taken per jar at 1, 2, 4, 7, and 9.5 mm, being careful to keep a minimum of 3 mm between penetration sites and the side of the jar. All samples were measured at a temperature of 23±2° C. and 50±5% relative humidity unless otherwise stated. A curve of force versus probe depth is obtained. Curves from replicate measurements on a single cleansing balm are averaged, and the average force at 1, 2, 4, 7 and 9.5 mm is obtained. The values of force across these depths represents the Hardness Profile, which can have a maximum at any depth. The maximum minus the minimum values across these depths are defined as the Hardness Range and the average of the values at these 5 depths is considered the Average Hardness. The ratio of the maximum hardness between 1.0 and 4.0 mm to the hardness at 9.5 mm is defined as the skin to bulk hardness ratio.

### Oil Binding Capacity Test Method

The Oil Binding Capacity Test Method is used to measure the fraction of the composition retained under conditions of centrifugation. Preweighed sample compositions are each placed in a centrifuge equipped with a filter and subjected to 800 G of centrifugal force for 20 minutes, and the mass of each specimen passing through the filter and collected in the tube is then measured, from which each retained specimen fraction is calculated. This method is carried out in an environment 23±2° C. and 50±5% relative humidity environment unless otherwise specified, and all materials and apparatus used are allowed to equilibrate to lab conditions for at least two hours prior to use.

At least two specimens, preferably three specimens, of a single cleansing balm composition are collected and individually analyzed. A transfer tube (such as part number 190195P, Repligen Corporation, Boston, MA, USA, or equivalent) is used to harvest each specimen, 0.75+0.25 g in mass. Each specimen plug of sample composition in the transfer tube is pushed into a preweighed filter unit of a filter centrifuge tube (Spin-X-Mfr #8169 Costar, Inc., Corning, NY, USA or equivalent) and the mass of the specimen is determined by difference and recorded to the nearest 0.0001 g. Each centrifuge tube is separately preweighed from the loaded filter unit, and its mass is recorded to the nearest 0.0001 g. Each loaded filter unit and centrifuge tube assembly containing a specimen is subjected to centrifugation at 800 G for 20.0 minutes. (One example of suitable centrifuge apparatus and RPM setting to achieve this is the Sorvall Legend Micro 21, Thermo Fisher Scientific, USA, operated at 3000 RPM). Immediately after centrifugation, the filter unit is removed from each filter-unit-and-centrifuge-tube assembly, and the mass of the centrifuge tube and containing expressed product composition is recorded to the nearest 0.0001 g, from which the mass of product expressed from each specimen is calculated by difference to the nearest 0.0001 g. The mass of specimen retained in the filter is defined as the oil binding capacity and defined for each specimen as:

$$specimen oil binding capacity [ \% ] = 100 \% \times ( 1 - \frac{mass of expressed product composition [ g ]}{mass of specimen [ g ]} ) .$$

The arithmetic mean of the replicate specimen oil binding capacity is calculated to the nearest 0.1% and reported as the Oil Binding Capacity of the cleansing balm.

### Examples

The following data and examples are provided to help illustrate the skincare compositions described herein. The exemplified compositions are given solely for the purpose of illustration and are not to be construed as limitations of the present disclosure, as many variations thereof are possible without departing from the spirit and scope of the disclosure. All parts, percentages, and ratios herein are by weight unless otherwise specified.

Examples 1-30, 34-36, and 39-64 were made according to the method described herein. Examples 31, 37, and 38 could be made according to the method described herein.

The Examples were considered preferred if the following criteria were met:
The examples passed a qualitative stability observation and ease of use observation. To pass the qualitative observation the composition needs to be phase stable without weeping oil by visual detection including weeping oil immediately after the product is scooped. The composition passed the case of use if it could be scooped from the jar with normal pressure as a creamy balm and had consistent texture upon visual inspection. If the sample was too hard to easily scoop, fractured when scooped, or was too runny (e.g., felt like a traditional body lotion dispensed from a pump dispenser), it did not pass the case of use test. In many instances, if the samples did not pass the qualitative stability observations, no additional testing was performed.
The average hardness was between 50-350 gf, according to the Penetration Test, described herein.
The skin: bulk hardness ratio greater than 0.60 and less than 2.0, according to the Penetration Test, described herein.
The % oil binding capacity is >65%, according to the Oil Binding Capacity Test Method, described herein.

Examples 1-22 are not preferred. In Examples 8-10, 12, 13, and 16-22 the oil separated, and the product was too soft. This indicates that the combination of wax structurants did not crystalize enough to hold the oil. Examples 6-7 and 11 had no visible oil separation, however they were too hard to be consumer acceptable. This indicates that the combination of waxes crystalized too much. When the product is too hard, it can often have lower oil binding and leak oil during storage or use. Examples 2-3, 5, and 14-15 all had an uneven texture during spreading, which also indicates that the combination of waxes was not consumer preferred.

Examples 23-26, 29, 32-36, 41-43, 45, and 47-67 may be preferred compositions since these examples had no oil separation, good harness, and a smooth texture. These examples had a combination of Emulium® Mellifera MB (Gattefossé®), stearyl behenate, tribehenin, and C18-36 acid triglyceride, indicating that this is a combination of wax structurants and oils could be used in consumer preferred cleansing balm products.

It is believed that other combinations of oils, wax structurants, and emulsifiers with similar properties could be used to create consumer acceptable cleansing balm compositions. For example, Examples 47, Examples 47, 49, 53, 54, 57 contain 2-8.8% shea butter as a wax structurant and Examples 59-60 contained 3.3-6.6% hydrogenated rapeseed oil as a wax structurant. Examples 52, 55-56, 58, and 61-64 contained 0.5-1.5% behenyl alcohol, as an additional structurant. However, as the not preferred examples illustrate, it can be difficult to formulate an effective cleansing balm with the consumer preferred physical parameters.

Example 27 is not consumer preferred because it was too soft and the oil separated. Example 27 is similar to Example 23. The main difference is that Example 27 had 3% behenyl behenate, while Example 23 had stearyl behenate. This indicates that behenyl behenate and stearyl behenate do not behave the same in cleansing balm compositions and additional experiments were needed to make a consumer preferred composition with behenyl behenate wax structurant.

Example 28 is not consumer preferred because it was too soft, and the oil separated. Example 28 is similar to Examples 23-25. The main difference is that Example 28 includes >50% triheptanoin in combination with other oils and Examples 23-25 includes >50% caprylic/capric triglyceride. This indicates that triheptanoin and caprylic/capric triglyceride are not interchangeable.

Example 31 is not consumer preferred because it was too soft the oil separated. Example 31 contains 3% of Emulium® Mellifera MB, which indicates that it can be advantageous to formulate with a higher level of Emulium® Mellifera MB.

Examples 39, 40, and 44 were not consumer preferred because the oil separated. These examples all contained hydrogenated castor oil. It may be preferable to formulate without hydrogenated castor oil in these cleansing balm compositions.

Example 46 was not consumer preferred because the oil separated. Example 46 is similar to Example 45; however, the emulsifier is different. Example 46 had 8% polyglyceryl-6 myristate and Example 45 had 8% polyglyceryl-6 dicaprate. This indicates that polyglyceryl-6 myristate and polyglyceryl-6 dicaprate are not interchangeable.

### Examples

TABLE 1

| | Ex. 1 | Ex. 2 | Ex. 3 | Ex. 4 | Ex. 5 | Ex. 6 | Ex. 7 | Ex. 8 |
|---|---|---|---|---|---|---|---|---|
| Emollient (Oil) | | | | | | | | |
| Cetyl Ethyl | 71 | 11 | 11 | 11 | 11 | 11 | 11 | 11 |
| Hexanoate | | | | | | | | |
| Cetyl Ethyl | | | | | | | | |
| Hexanoate | | | | | | | | |
| Coco- | | 11 | 11 | 11 | 11 | 11 | 11 | 11 |
| Caprylate/Caprate | | | | | | | | |
| Caprylic/Capric | 8 | 57.0 | 50.0 | 57.0 | 50.0 | 54.0 | 51.0 | 54.8 |
| Triglyceride | | | | | | | | |
| Triheptanoin | | | | | | | | |
| Emulsifier | | | | | | | | |
| PEG-20 Glyceryl | | | | | | | | |
| Triisostearate¹ | | | | | | | | |
| PEG-10 Isostearate² | | | | | | | | |
| Sorbitan | | | | | | | | |
| Sesquioleate³ | | | | | | | | |
| Polyglyceryl-6 | 6 | 6 | 6 | 6 | 6 | 6 | 6 | 6 |
| Distearate (and) | | | | | | | | |
| Jojoba Esters (and) | | | | | | | | |
| Polyglyceryl-3 | | | | | | | | |
| Beeswax (and) | | | | | | | | |
| Cetyl Alcohol⁴ | | | | | | | | |
| Sorbeth-30 | 8 | 8 | 8 | 8 | 8 | 8 | 8 | 8 |
| Tetraoleate⁵ | | | | | | | | |
| Glyceryl laurate⁶ | | | | | | | | |
| Polyglyceryl-10 | | | | | | | | |
| Dioleate⁷ | | | | | | | | |
| Polyglyceryl-6 | | | | | | | | |
| Dicaprate⁸ | | | | | | | | |
| Glyceryl | | | | | | | | |
| Citrate/Lactate/ | | | | | | | | |
| Linoleate/Oleate, | | | | | | | | |
| Polyglyceryl-4 | | | | | | | | |
| Cocoate, | | | | | | | | |
| Polyglyceryl-3 | | | | | | | | |
| Caprate, Glyceryl | | | | | | | | |
| Caprylate⁹ | | | | | | | | |
| Polyglyceryl-6 | | | | | | | | |
| Oleate, Potassium | | | | | | | | |
| Olivate¹⁰ | | | | | | | | |
| Structurant (Wax) | | | | | | | | |
| Oryza Sativa (Rice) | 1 | 1 | 1 | 1 | 1 | 1 | 1 | 0.2 |
| Bran Wax¹¹ | | | | | | | | |
| Behenyl Behenate¹² | 6 | 6 | 6 | | | | | |
| Stearyl Behenate¹³ | | | | 6 | 6 | 9 | 12 | |
| Tribehenin¹⁴ | | | | | | | | 9 |
| Shea Butter¹⁵ | | | 2 | | 2 | | | |
| Hydrogenated | | | | | | | | |
| Rapeseed oil¹⁶ | | | | | | | | |
| Hydrogenated | | | | | | | | |
| Castor Oil¹⁷ | | | | | | | | |
| C18-36 Acid | | | | | | | | |
| Triglyceride¹⁸ | | | | | | | | |
| Tapioca starch¹⁹ | | | 5 | | 5 | | | |
| Botanical Extracts | | | | | | | | |
| & Essential Oils | | | | | | | | |
| Phenoxyethanol | | | | | | | | |
| Stability | Liquid | Liquid separates | | Liquid | No separation | | | Liquid |
| Observation | separates | during use | | seperates | | | | seperates |
| (weeping of oil) | | | | | | | | |
| Ease of Use | | Uneven | Uneven | | Uneven | Too | Too | Too |
| Observation | | texture | texture | | texture | Hard | Hard | Soft |
| (scooping & | | during | during | | during | | | |
| spreading of balm) | | use | use | | use | | | |
| Hardness Data (gf) | | | | | | | | |
| Probe Depth in | | | | | | | | |
| Sample (mm) | | | | | | | | |
| 1.0 | 120 | 240 | 111 | 54 | 104 | 324 | 353 | 2 |
| 2.0 | 169 | 362 | 193 | 102 | 198 | 484 | 506 | 3 |
| 4.0 | 183 | 380 | 286 | 147 | 279 | 680 | 607 | 4 |
| 7.0 | 211 | 363 | 367 | 174 | 344 | 764 | 655 | 6 |
| 9.5 | 261 | 436 | 516 | 187 | 376 | 601 | 710 | 9 |
| Average Hardness | 188.7 | 356.2 | 294.8 | 132.7 | 260.2 | 570.5 | 565.9 | 4.9 |
| Skin:Bulk | 0.70 | 0.87 | 0.55 | 0.79 | 0.74 | 1.13 | 0.86 | 0.48 |
| Hardness Ratio | | | | | | | | |
| Oil Binding | | | 70.6 | | | | | |
| Capacity (%) | | | | | | | | |

| | |
|---|---|
| Overall Conclusion | Not preferred |

TABLE 2

| | Ex. 9 | Ex. 10 | Ex. 11 | Ex. 12 | Ex. 13 | Ex. 14 | Ex. 15 | Ex. 16 |
|---|---|---|---|---|---|---|---|---|
| Emollient (Oil) | | | | | | | | |
| Cetyl Ethyl | 11 | 11 | 11 | 11 | 11 | 11 | 11 | 11 |
| Hexanoate | | | | | | | | |
| Cetyl Ethyl | | | | | | | | |
| Hexanoate | | | | | | | | |
| Coco- | 11 | 11 | 11 | 11 | 11 | 11 | 11 | 11 |
| Caprylate/Caprate | | | | | | | | |
| Caprylic/Capric | 51.8 | 54.0 | 51.8 | 52.0 | 52.0 | 52.0 | 50.0 | 48.0 |
| Triglyceride | | | | | | | | |
| Triheptanoin | | | | | | | | |
| Emulsifier | | | | | | | | |
| PEG-20 Glyceryl | | | | | | | | |
| Triisostearate¹ | | | | | | | | |
| PEG-10 | | | | | | | | |
| Isostearate² | | | | | | | | |
| Sorbitan | | | | | | | | |
| Sesquioleate³ | | | | | | | | |
| Polyglyceryl-6 | 6 | 6 | 6 | 6 | 6 | 6 | 6 | 6 |
| Distearate (and) | | | | | | | | |
| Jojoba Esters (and) | | | | | | | | |
| Polyglyceryl-3 | | | | | | | | |
| Beeswax (and) | | | | | | | | |
| Cetyl Alcohol⁴ | | | | | | | | |
| Sorbeth-30 | 8 | 8 | 8 | 8 | 8 | 8 | 8 | 8 |
| Tetraoleate⁵ | | | | | | | | |
| Glyceryl laurate⁶ | | | | | | | | |
| Polyglyceryl-10 | | | | | | | | |
| Dioleate⁷ | | | | | | | | |
| Polyglyceryl-6 | | | | | | | | |
| Dicaprate⁸ | | | | | | | | |
| Glyceryl | | | | | | | | |
| Citrate/Lactate/ | | | | | | | | |
| Linoleate/Oleate, | | | | | | | | |
| Polyglyceryl-4 | | | | | | | | |
| Cocoate, | | | | | | | | |
| Polyglyceryl-3 | | | | | | | | |
| Caprate, Glyceryl | | | | | | | | |
| Caprylate⁹ | | | | | | | | |
| Polyglyceryl-6 | | | | | | | | |
| Oleate, Potassium | | | | | | | | |
| Olivate¹⁰ | | | | | | | | |
| Structurant (Wax) | | | | | | | | |
| Oryza Sativa (Rice) | 0.2 | 1 | 0.2 | | | | | |
| Bran Wax¹¹ | | | | | | | | |
| Behenyl Behenate¹² | | | | | | | | |
| Stearyl Behenate¹³ | | | 6 | 3 | 1 | 4 | 5 | 3 |
| Tribehenin¹⁴ | 12 | 9 | 6 | 9 | 11 | 8 | 9 | 8 |
| Shea Butter¹⁵ | | | | | | | | 5 |
| Hydrogenated | | | | | | | | |
| Rapeseed oil¹⁶ | | | | | | | | |
| Hydrogenated | | | | | | | | |
| Castor Oil¹⁷ | | | | | | | | |
| C18-36 Acid | | | | | | | | |
| Triglyceride¹⁸ | | | | | | | | |
| Tapioca starch¹⁹ | | | | | | | | |
| Botanical Extracts | | | | | | | | |
| & Essential Oils | | | | | | | | |
| Phenoxyethanol | | | | | | | | |
| Stability | Liquid separates | | No | Liquid separates | | | | |
| Observation | | | separation | | | | | |
| (weeping of oil) | | | | | | | | |
| Ease of Use | Too | Too | Too | Too | Too | Uneven texture | | Too |
| Observation | Soft | Soft | Hard | Soft | Soft | during use | | Soft |
| (scooping & | | | | | | | | |
| spreading of balm) | | | | | | | | |
| Hardness Data (gf) | | | | | | | | |
| Probe Depth in | | | | | | | | |
| Sample (mm) | | | | | | | | |
| 1.0 | 1 | 16 | 218 | 1 | 1 | 177 | 23 | 5 |
| 2.0 | 1 | 34 | 373 | 2 | 2 | 271 | 99 | 10 |
| 4.0 | 1 | 47 | 419 | 4 | 3 | 305 | 290 | 18 |
| 7.0 | 4 | 57 | 451 | 5 | 3 | 317 | 376 | 20 |
| 9.5 | 4 | 58 | 524 | 6 | 3 | 302 | 395 | 17 |
| Average Hardness | 2.0 | 42.5 | 397.1 | 3.6 | 2.5 | 274.3 | 236.5 | 14.0 |
| Skin:Bulk | 0.25 | 0.81 | 0.80 | 0.62 | 0.85 | 1.01 | 0.73 | 1.10 |
| Hardness Ratio | | | | | | | | |
| Oil Binding | | | 94.3 | 48.6 | | | | |
| Capacity (%) | | | | | | | | |

| | |
|---|---|
| Overall Conclusion | Not Preferred |

TABLE 3

| | Ex. 17 | Ex. 18 | Ex. 19 | Ex. 20 | Ex. 21 | Ex. 22 | Ex. 23 | Ex. 24 |
|---|---|---|---|---|---|---|---|---|
| Emollient (Oil) | | | | | | | | |
| Cetyl Ethyl | 11 | 11 | 11 | 11 | 11 | 11 | 11 | 11 |
| Hexanoate | | | | | | | | |
| Cetyl Ethyl | | | | | | | | |
| Hexanoate | | | | | | | | |
| Coco- | 11 | 11 | 11 | 11 | 11 | 11 | 11 | 11 |
| Caprylate/Caprate | | | | | | | | |
| Caprylic/Capric | 48.0 | 53.0 | 54.0 | 53.0 | 50.8 | 53.39 | 52.89 | 52.34 |
| Triglyceride | | | | | | | | |
| Triheptanoin | | | | | | | | |
| Emulsifier | | | | | | | | |
| PEG-20 Glyceryl | | | | | | | | |
| Triisostearate¹ | | | | | | | | |
| PEG-10 | | | | | | | | |
| Isostearate² | | | | | | | | |
| Sorbitan | | | | | | | | |
| Sesquioleate³ | | | | | | | | |
| Polyglyceryl-6 | 6 | 6 | 6 | 6 | 6 | 6 | 6 | 6 |
| Distearate (and) | | | | | | | | |
| Jojoba Esters (and) | | | | | | | | |
| Polyglyceryl-3 | | | | | | | | |
| Beeswax (and) | | | | | | | | |
| Cetyl Alcohol⁴ | | | | | | | | |
| Sorbeth-30 | 8 | 8 | 8 | 8 | 8 | 8 | 8 | 8 |
| Tetraoleate⁵ | | | | | | | | |
| Glyceryl laurate⁶ | | | | | | | | |
| Polyglyceryl-10 | | | | | | | | |
| Dioleate⁷ | | | | | | | | |
| Polyglyceryl-6 | | | | | | | | |
| Dicaprate⁸ | | | | | | | | |
| Glyceryl | | | | | | | | |
| Citrate/Lactate/ | | | | | | | | |
| Linoleate/Oleate, | | | | | | | | |
| Polyglyceryl-4 | | | | | | | | |
| Cocoate, | | | | | | | | |
| Polyglyceryl-3 | | | | | | | | |
| Caprate, Glyceryl | | | | | | | | |
| Caprylate⁹ | | | | | | | | |
| Polyglyceryl-6 | | | | | | | | |
| Oleate, Potassium | | | | | | | | |
| Olivate¹⁰ | | | | | | | | |
| Structurant (Wax) | | | | | | | | |
| Oryza Sativa (Rice) | | | | | | | | |
| Bran Wax¹¹ | | | | | | | | |
| Behenyl Behenate¹² | | | | | | | | |
| Stearyl Behenate¹³ | | 3 | 3 | 3.30 | 3.96 | 3.5 | 3 | 3 |
| Tribehenin¹⁴ | 8 | 8 | 7 | 7.70 | 9.24 | 7 | 7 | 7 |
| Shea Butter¹⁵ | 5 | | | | | | | |
| Hydrogenated | 3 | | | | | | | |
| Rapeseed oil¹⁶ | | | | | | | | |
| Hydrogenated | | | | | | | | |
| Castor Oil¹⁷ | | | | | | | | |
| C18-36 Acid | | | | | | | 1 | 1.5 |
| Triglyceride¹⁸ | | | | | | | | |
| Tapioca starch¹⁹ | | | | | | | | |
| Botanical Extracts | | | | | | 0.11 | 0.11 | 0.16 |
| & Essential Oils | | | | | | | | |
| Phenoxyethanol | | | | | | | | |
| Stability | Liquid separates | | | | | | No separation | |
| Observation | | | | | | | | |
| (weeping of oil) | | | | | | | | |
| Ease of Use | Too | Too | Too | Too | Too | Too | Good Hardness - | |
| Observation | Soft | Soft | Soft | Soft | Soft | Soft | Smooth | |
| (scooping & | | | | | | | Texture | |
| spreading of balm) | | | | | | | | |
| Hardness Data (gf) | | | | | | | | |
| Probe Depth in | | | | | | | | |
| Sample (mm) | | | | | | | | |
| 1.0 | 1 | 3 | 8 | 6 | 10 | 12 | 69 | 93 |
| 2.0 | 1 | 5 | 14 | 11 | 20 | 23 | 88 | 125 |
| 4.0 | 2 | 6 | 16 | 18 | 41 | 37 | 92 | 159 |
| 7.0 | 2 | 6 | 18 | 20 | 41 | 45 | 162 | 215 |
| 9.5 | 2 | 6 | 23 | 17 | 33 | 45 | 172 | 215 |
| Average Hardness | 1.7 | 5.0 | 16.1 | 14.6 | 28.9 | 32.4 | 116.8 | 161.3 |
| Skin:Bulk | 0.96 | 0.95 | 0.71 | 1.05 | 1.24 | 0.84 | 0.54 | 0.74 |
| Hardness Ratio | | | | | | | | |
| Oil Binding | | | 56.5 | | | | 86.1 | 89.1 |
| Capacity (%) | | | | | | | | |

| | | |
|---|---|---|
| Overall Conclusion | Not preferred | Preferred |

TABLE 4

| | Ex. 25 | Ex. 26 | Ex. 27 | Ex. 28 | Ex. 29 | Ex. 30 | Ex. 31 | Ex. 32 |
|---|---|---|---|---|---|---|---|---|
| Emollient (Oil) | | | | | | | | |
| Cetyl Ethyl | 11 | | | | | | | |
| Hexanoate | | | | | | | | |
| Cetyl Ethyl | | 11 | 11 | 11 | 11 | 11 | 11 | 11 |
| Hexanoate | | | | | | | | |
| Coco- | 11 | 11 | 11 | 11 | 11 | 11 | 11 | 11 |
| Caprylate/Caprate | | | | | | | | |
| Caprylic/Capric | 51.84 | 52.69 | 52.44 | | 47.44 | 51.6 | 55.44 | 49.44 |
| Triglyceride | | | | | | | | |
| Triheptanoin | | | | 52.44 | | | | |
| Emulsifier | | | | | | | | |
| PEG-20 Glyceryl | | | | | | | | |
| Triisostearate¹ | | | | | | | | |
| PEG-10 | | | | | | | | |
| Isostearate² | | | | | | | | |
| Sorbitan | | | | | | 0.35 | | |
| Sesquioleate³ | | | | | | | | |
| Polyglyceryl-6 | 6 | 6 | 6 | 6 | 6 | 6 | 3 | 9 |
| Distearate (and) | | | | | | | | |
| Jojoba Esters (and) | | | | | | | | |
| Polyglyceryl-3 | | | | | | | | |
| Beeswax (and) | | | | | | | | |
| Cetyl Alcohol⁴ | | | | | | | | |
| Sorbeth-30 | 8 | 8 | 8 | 8 | 13 | | 8 | 8 |
| Tetraoleate⁵ | | | | | | | | |
| Glyceryl laurate⁶ | | | | | | 0.1 | | |
| Polyglyceryl-10 | | | | | | 4.25 | | |
| Dioleate⁷ | | | | | | | | |
| Polyglyceryl-6 | | | | | | 2.3 | | |
| Dicaprate⁸ | | | | | | | | |
| Glyceryl | | | | | | 0.85 | | |
| Citrate/Lactate/ | | | | | | | | |
| Linoleate/Oleate, | | | | | | | | |
| Polyglyceryl-4 | | | | | | | | |
| Cocoate, | | | | | | | | |
| Polyglyceryl-3 | | | | | | | | |
| Caprate, Glyceryl | | | | | | | | |
| Caprylate⁹ | | | | | | | | |
| Polyglyceryl-6 | | | | | | 1 | | |
| Oleate, Potassium | | | | | | | | |
| Olivate¹⁰ | | | | | | | | |
| Structurant (Wax) | | | | | | | | |
| Oryza Sativa (Rice) | | | | | | | | |
| Bran Wax¹¹ | | | | | | | | |
| Behenyl Behenate¹² | | | 3 | | | | | |
| Stearyl Behenate¹³ | 3 | 3 | | 3 | 3 | 3 | 3 | 3 |
| Tribehenin¹⁴ | 7 | 7 | 7 | 7 | 7 | 7 | 7 | 7 |
| Shea Butter¹⁵ | | | | | | | | |
| Hydrogenated | | | | | | | | |
| Rapeseed oil¹⁶ | | | | | | | | |
| Hydrogenated | | | | | | | | |
| Castor Oil¹⁷ | | | | | | | | |
| C18-36 Acid | 2 | 0.75 | 1 | 1 | 1 | 1 | 1 | 1 |
| Triglyceride¹⁸ | | | | | | | | |
| Tapioca starch¹⁹ | | | | | | | | |
| Botanical Extracts | 0.16 | 0.56 | 0.56 | 0.56 | 0.56 | 0.56 | 0.56 | 0.56 |
| & Essential Oils | | | | | | | | |
| Phenoxyethanol | | | | | | | | |
| Stability | No separation | | Liquid separates | | No | | Liquid | No |
| Observation | | | | | Separation | | separates | separation |
| (weeping of oil) | | | | | | | | |
| Ease of Use | Good Hardness - | | Too | Too | Good | | | Good |
| Observation | Smooth | | Soft | Soft | Hardness - | | | Hardness - |
| (scooping & | Texture | | | | Smooth | | | Smooth |
| spreading of balm) | | | | | Texture | | | Texture |
| Hardness Data (gf) | | | | | | | | |
| Probe Depth in | | | | | | | | |
| Sample (mm) | | | | | | | | |
| 1.0 | 88 | 65 | 15 | 26 | 55 | | 16 | 127 |
| 2.0 | 118 | 84 | 23 | 35 | 82 | | 23 | 180 |
| 4.0 | 139 | 108 | 31 | 38 | 114 | | 23 | 257 |
| 7.0 | 209 | 122 | 43 | 44 | 128 | | 25 | 318 |
| 9.5 | 225 | 146 | 55 | 52 | 121 | | 32 | 310 |
| Average Hardness | 156.0 | 105.0 | 33.2 | 38.9 | 100.2 | | 23.7 | 238.4 |
| Skin:Bulk | 0.62 | 0.74 | 0.56 | 0.74 | 0.94 | | 0.74 | 0.83 |
| Hardness Ratio | | | | | | | | |
| Oil Binding | 92.8 | 79.9 | 58.9 | 69.3 | 86.8 | | 43.1 | 81.9 |
| Capacity (%) | | | | | | | | |

| | | | | | |
|---|---|---|---|---|---|
| Overall Conclusion | Preferred | Not preferred | Preferred | Not | Preferred |

preferred

TABLE 5

| | Ex. 33 | Ex. 34 | Ex. 35 | Ex. 36 | Ex. 37 | Ex. 38 | Ex. 39 | Ex. 40 |
|---|---|---|---|---|---|---|---|---|
| Emollient (Oil) | | | | | | | | |
| Cetyl Ethyl | | | | | | | | |
| Hexanoate | | | | | | | | |
| Cetyl Ethyl | 11 | 11 | 11 | 11 | 11 | 11 | 11 | 11 |
| Hexanoate | | | | | | | | |
| Coco- | 11 | 11 | 11 | 11 | 11 | 11 | 11 | 11 |
| Caprylate/Caprate | | | | | | | | |
| Caprylic/Capric | 50.44 | 54.44 | 50.44 | 54.44 | 55.84 | 52.44 | 52.44 | 49.44 |
| Triglyceride | | | | | | | | |
| Triheptanoin | | | | | | | | |
| Emulsifier | | | | | | | | |
| PEG-20 Glyceryl | | | | | | | | |
| Triisostearate¹ | | | | | | | | |
| PEG-10 | | | | | | | | |
| Isostearate² | | | | | | | | |
| Sorbitan | | | | | 0.5 | 1 | | |
| Sesquioleate³ | | | | | | | | |
| Polyglyceryl-6 | 6 | 6 | 6 | 6 | 6 | 6 | 6 | 6 |
| Distearate (and) | | | | | | | | |
| Jojoba Esters (and) | | | | | | | | |
| Polyglyceryl-3 | | | | | | | | |
| Beeswax (and) | | | | | | | | |
| Cetyl Alcohol⁴ | | | | | | | | |
| Sorbeth-30 | 8 | 8 | 8 | 8 | | | 8 | 8 |
| Tetraoleate⁵ | | | | | | | | |
| Glyceryl laurate⁶ | | | | | 0.1 | | | |
| Polyglyceryl-10 | | | | | | | | |
| Dioleate⁷ | | | | | | | | |
| Polyglyceryl-6 | | | | | | | | |
| Dicaprate⁸ | | | | | | | | |
| Glyceryl | | | | | 2 | 3.5 | | |
| Citrate/Lactate/ | | | | | | | | |
| Linoleate/Oleate, | | | | | | | | |
| Polyglyceryl-4 | | | | | | | | |
| Cocoate, | | | | | | | | |
| Polyglyceryl-3 | | | | | | | | |
| Caprate, Glyceryl | | | | | | | | |
| Caprylate⁹ | | | | | | | | |
| Polyglyceryl-6 | | | | | 2 | 3.5 | | |
| Oleate, Potassium | | | | | | | | |
| Olivate¹⁰ | | | | | | | | |
| Structurant (Wax) | | | | | | | | |
| Oryza Sativa (Rice) | | | | | | | | |
| Bran Wax¹¹ | | | | | | | | |
| Behenyl Behenate¹² | | | | | | | | |
| Stearyl Behenate ¹³ | 5 | 1 | 3 | 3 | 3 | 3 | | |
| Tribehenin¹⁴ | 7 | 7 | 9 | 5 | 7 | 7 | 7 | 7 |
| Shea Butter¹⁵ | | | | | | | | |
| Hydrogenated | | | | | | | | |
| Rapeseed oil¹⁶ | | | | | | | | |
| Hydrogenated | | | | | | | 3 | 6 |
| Castor Oil¹⁷ | | | | | | | | |
| C18-36 Acid | 1 | 1 | 1 | 1 | 1 | 1 | 1 | 1 |
| Triglyceride¹⁸ | | | | | | | | |
| Tapioca starch¹⁹ | | | | | | | | |
| Botanical Extracts | 0.56 | 0.56 | 0.56 | 0.56 | 0.56 | 0.56 | 0.56 | 0.56 |
| & Essential Oils | | | | | | | | |
| Phenoxyethanol | | | | | | | | |
| Stability | No separation | | | | | | Liquid Separates | |
| Observation | | | | | | | | |
| (weeping of oil) | | | | | | | | |
| Ease of Use | Good Hardness - Smooth Texture | | | | | | Too Soft | |
| Observation | | | | | | | | |
| (scooping & | | | | | | | | |
| spreading of balm) | | | | | | | | |
| Hardness Data (gf) | | | | | | | | |
| Probe Depth in | | | | | | | | |
| Sample (mm) | | | | | | | | |
| 1.0 | 50 | 42 | 65 | 60 | | | 15 | 12 |
| 2.0 | 73 | 63 | 85 | 83 | | | 23 | 19 |
| 4.0 | 89 | 83 | 93 | 82 | | | 22 | 18 |
| 7.0 | 105 | 95 | 102 | 102 | | | 26 | 20 |
| 9.5 | 125 | 94 | 137 | 139 | | | 34 | 30 |
| Average Hardness | 88.4 | 75.4 | 96.4 | 93.1 | | | 23.9 | 19.7 |
| Skin:Bulk | 0.71 | 0.89 | 0.68 | 0.59 | | | 0.67 | 0.63 |
| Hardness Ratio | | | | | | | | |
| Oil Binding | 71.4 | 79.5 | 75.5 | 82.7 | | | 61.9 | 49.0 |
| Capacity (%) | | | | | | | | |

| | | |
|---|---|---|
| Overall Conclusion | Preferred | Not preferred |

TABLE 6

| | Ex. 41 | Ex. 42 | Ex. 43 | Ex. 44 | Ex. 45 | Ex. 46 | Ex. 47 | Ex. 48 |
|---|---|---|---|---|---|---|---|---|
| Emollient (oil) | | | | | | | | |
| Cetyl Ethyl | | | | | | | | |
| Hexanoate | | | | | | | | |
| Cetyl Ethyl | 11 | 11 | 11 | 11 | 11 | 11 | 11 | 11 |
| Hexanoate | | | | | | | | |
| Coco- | 11 | 11 | 11 | 11 | 11 | 11 | 11 | 11 |
| Caprylate/Caprate | | | | | | | | |
| Caprylic/Capric | 47.44 | 51.44 | 49.44 | 49.44 | 52.44 | 52.44 | 50.44 | 52.44 |
| Triglyceride | | | | | | | | |
| Triheptanoin | | | | | | | | |
| Emulsifier | | | | | | | | |
| PEG-20 Glyceryl | | | | | | | | |
| Triisostearate¹ | | | | | | | | |
| PEG-10 | | | | | | | | |
| Isostearate² | | | | | | | | |
| Sorbitan | | | | | | | | |
| Sesquioleate³ | | | | | | | | |
| Polyglyceryl-6 | 6 | 6 | 9 | 6 | 6 | 6 | 6 | 4 |
| Distearate (and) | | | | | | | | |
| Jojoba Esters (and) | | | | | | | | |
| Polyglyceryl-3 | | | | | | | | |
| Beeswax (and) | | | | | | | | |
| Cetyl Alcohol⁴ | | | | | | | | |
| Sorbeth-30 | 13 | 8 | 8 | 8 | | | 8 | 8 |
| Tetraoleate⁵ | | | | | | | | |
| Glyceryl laurate⁶ | | | | | | | | |
| Polyglyceryl-10 | | | | | | | | |
| Dioleate⁷ | | | | | | | | |
| Polyglyceryl-6 | | | | | 8 | | | |
| Dicaprate⁸ | | | | | | | | |
| Polyglyceryl-6 | | | | | | 8 | | |
| Myristate² | | | | | | | | |
| Glyceryl | | | | | | | | |
| Citrate/Lactate/ | | | | | | | | |
| Linoleate/Oleate, | | | | | | | | |
| Polyglyceryl-4 | | | | | | | | |
| Cocoate, | | | | | | | | |
| Polyglyceryl-3 | | | | | | | | |
| Caprate, Glyceryl | | | | | | | | |
| Caprylate⁹ | | | | | | | | |
| Polyglyceryl-6 | | | | | | | | |
| Oleate, Potassium | | | | | | | | |
| Olivate¹⁰ | | | | | | | | |
| Structurant (wax) | | | | | | | | |
| Oryza Sativa (Rice) | | | | | | | | |
| Bran Wax¹¹ | | | | | | | | |
| Behenyl Behenate¹² | 3 | 3 | 3 | 3 | | | | |
| Stearyl Behenate¹³ | | | | | 3 | 3 | 3 | 3 |
| Tribehenin¹⁴ | 7 | 7 | 7 | 7 | 7 | 7 | 7 | 7 |
| Shea Butter¹⁵ | | | | | | | 2 | |
| Hydrogenated | | | | | | | | |
| Rapeseed oil¹⁶ | | | | | | | | |
| Hydrogenated | | | | 3 | | | | |
| Castor Oil¹⁷ | | | | | | | | |
| C18-36 Acid | 1 | 2 | 1 | 1 | 1 | 1 | 1 | 3 |
| Triglyceride¹⁸ | | | | | | | | |
| Tapioca starch¹⁹ | | | | | | | | |
| Botanical Extracts | 0.56 | 0.56 | 0.56 | 0.56 | 0.56 | 0.56 | 0.56 | 0.56 |
| & Essential Oils | | | | | | | | |
| Phenoxyethanol | | | | | | | | |
| Stability | No separation | | | Liquid | No | Liquid | No separation | |
| Observation | | | | separates | separation | separates | | |
| (weeping of oil) | | | | over | | over | | |
| | | | | time | | time | | |
| Ease of Use | Appropriate hardness and | | | Too | Appropriate | | Appropriate | |
| Observation | smooth texture | | | soft | hardness | | hardness and | |
| (scooping & | | | | | and | | smooth texture | |
| spreading of balm) | | | | | smooth | | | |
| | | | | | texture | | | |
| Hardness Data (gf) | | | | | | | | |
| Probe Depth in | | | | | | | | |
| Sample (mm) | | | | | | | | |
| 1.0 | 51 | 58 | 202 | 13 | 170 | 34 | 63 | 32 |
| 2.0 | 82 | 88 | 289 | 20 | 220 | 55 | 91 | 47 |
| 4.0 | 126 | 120 | 388 | 25 | 341 | 81 | 88 | 56 |
| 7.0 | 150 | 139 | 421 | 31 | 340 | 96 | 129 | 63 |
| 9.5 | 140 | 148 | 401 | 39 | 306 | 109 | 140 | 69 |
| Average Hardness | 109.9 | 110.8 | 340.4 | 25.7 | 275.5 | 75.0 | 102.0 | 53.1 |
| Skin:Bulk | 0.90 | 0.81 | 0.97 | 0.64 | 1.11 | 0.75 | 0.65 | 0.81 |
| Hardness Ratio | | | | | | | | |
| Oil Binding | 63.4 | 73.9 | 79.9 | 59.4 | 87.4 | 49.7 | 79.4 | 73.8 |
| Capacity (%) | | | | | | | | |

| | | | | | |
|---|---|---|---|---|---|
| Overall Conclusion | Preferred | Not | Preferred | Not | Preferred |
| | | preferred | | preferred | |

TABLE 7

| | Ex. 49 | Ex. 50 | Ex. 51 | Ex. 52 | Ex. 53 | Ex. 54 | Ex. 55 | Ex. 56 |
|---|---|---|---|---|---|---|---|---|
| Emollient (oil) | | | | | | | | |
| Cetyl Ethyl | | | | | | | | |
| Hexanoate | | | | | | | | |
| Cetyl Ethyl | 11 | 11 | 11 | 11 | 11 | 11 | 11 | 11 |
| Hexanoate | | | | | | | | |
| Coco- | 11 | 11 | 11 | 11 | 11 | 11 | 11 | 11 |
| Caprylate/Caprate | | | | | | | | |
| Caprylic/Capric | 48.54 | 50.74 | 45.74 | 46.94 | 46.34 | 41.94 | 46.44 | 45.94 |
| Triglyceride | | | | | | | | |
| Triheptanoin | | | | | | | | |
| Emulsifier | | | | | | | | |
| PEG-20 Glyceryl | | | | | | | | |
| Triisostearate¹ | | | | | | | | |
| PEG-10 Isostearate² | | | | | | | | |
| Sorbitan | | | | | | | | |
| Sesquioleate³ | | | | | | | | |
| Polyglyceryl-6 | 6.6 | 6.6 | 6.6 | 6 | 6.6 | 6.6 | 6 | 6 |
| Distearate (and) | | | | | | | | |
| Jojoba Esters (and) | | | | | | | | |
| Polyglyceryl-3 | | | | | | | | |
| Beeswax (and) Cetyl | | | | | | | | |
| Alcohol⁴ | | | | | | | | |
| Sorbeth-30 | 8 | 8 | 13 | 13 | 8 | 8 | 13 | 13 |
| Tetraoleate⁵ | | | | | | | | |
| Glyceryl laurate⁶ | | | | | | | | |
| Polyglyceryl-10 | | | | | | | | |
| Dioleate⁷ | | | | | | | | |
| Polyglyceryl-6 | | | | | | | | |
| Dicaprate⁸ | | | | | | | | |
| Polyglyceryl-6 | | | | | | | | |
| Myristate²⁰ | | | | | | | | |
| Glyceryl | | | | | | | | |
| Citrate/Lactate/ | | | | | | | | |
| Linoleate/Oleate, | | | | | | | | |
| Polyglyceryl-4 | | | | | | | | |
| Cocoate, | | | | | | | | |
| Polyglyceryl-3 | | | | | | | | |
| Caprate, Glyceryl | | | | | | | | |
| Caprylate⁹ | | | | | | | | |
| Polyglyceryl-6 | | | | | | | | |
| Oleate, Potassium | | | | | | | | |
| Olivate¹⁰ | | | | | | | | |
| Structurant (wax) | | | | | | | | |
| Oryza Sativa (Rice) | | | | | | | | |
| Bran Wax¹¹ | | | | | | | | |
| Behenyl Behenate¹² | | | | | | | | |
| Stearyl Behenate¹³ | 3.3 | 3.3 | 3.3 | 3 | 3.3 | 3.3 | 3 | 3 |
| Tribehenin¹⁴ | 7.7 | 7.7 | 7.7 | 7 | 7.7 | 7.7 | 7 | 7 |
| Shea Butter¹⁵ | 2.2 | | | | 4.4 | 8.8 | | |
| Hydrogenated | | | | | | | | |
| Rapeseed oil¹⁶ | | | | | | | | |
| Hydrogenated | | | | | | | | |
| Castor Oil¹⁷ | | | | | | | | |
| C18-36 Acid | 1.1 | 1.1 | 1.1 | 1 | 1.1 | 1.1 | 1 | 1 |
| Triglyceride¹⁸ | | | | | | | | |
| Behenyl alcohol | | | | 0.5 | | | 1 | 1.5 |
| Tapioca starch¹⁹ | | | | | | | | |
| Botanical Extracts & | 0.56 | 0.56 | 0.56 | 0.56 | 0.56 | 0.56 | 0.56 | 0.56 |
| Essential Oils | | | | | | | | |
| Phenoxyethanol | | | | | | | | |
| Stability | No separation | | | | | | | |
| Observation | | | | | | | | |
| (weeping of oil) | | | | | | | | |
| Ease of Use | Appropriate hardness and smooth texture | | | | | | | |
| Observation | | | | | | | | |
| (scooping & | | | | | | | | |
| spreading of balm) | | | | | | | | |
| Hardness Data (gf) | | | | | | | | |
| Probe Depth in | | | | | | | | |
| Sample (mm) | | | | | | | | |
| 1.0 | 43 | 92 | 93 | 64 | 80 | 76 | 45 | 42 |
| 2.0 | 77 | 122 | 137 | 88 | 106 | 120 | 59 | 51 |
| 4.0 | 118 | 122 | 178 | 126 | 160 | 188 | 85 | 90 |
| 7.0 | 129 | 167 | 213 | 155 | 189 | 215 | 107 | 106 |
| 9.5 | 132 | 208 | 194 | 151 | 215 | 199 | 104 | 101 |
| Average Hardness | 99.7 | 142.2 | 162.9 | 116.7 | 150.0 | 159.7 | 79.9 | 77.9 |
| Skin:Bulk Hardness | 0.89 | 0.59 | 0.92 | 0.84 | 0.74 | 0.95 | 0.82 | 0.89 |
| Ratio | | | | | | | | |
| Oil Binding | 84.2 | 81.2 | 81.6 | 79.7 | 83.3 | 86.1 | 75.8 | 88.5 |
| Capacity (%) | | | | | | | | |

| | |
|---|---|
| Overall Conclusion | Preferred |

TABLE 8

| | Ex. 57 | Ex. 58 | Ex. 59 | Ex. 60 | Ex. 61 | Ex. 62 | Ex. 63 | Ex. 64 |
|---|---|---|---|---|---|---|---|---|
| Emollient (oil) | | | | | | | | |
| Cetyl Ethyl | | | | | | | | |
| Hexanoate | | | | | | | | |
| Cetyl Ethyl | 11 | 11 | 11 | 11 | 11 | 11 | 11 | 11 |
| Hexanoate | | | | | | | | |
| Coco- | 11 | 11 | 11 | 11 | 11 | 11 | 11 | 11 |
| Caprylate/Caprate | | | | | | | | |
| Caprylic/Capric | 49.14 | 50.84 | 50.74 | 47.44 | 46.44 | 46.44 | 46.34 | 46.24 |
| Triglyceride | | | | | | | | |
| Triheptanoin | | | | | | | | |
| Emulsifier | | | | | | | | |
| PEG-20 Glyceryl | | | | | | | | |
| Triisostearate¹ | | | | | | | | |
| PEG-10 Isostearate² | | | | | | | | |
| Sorbitan | | | | | | | | |
| Sesquioleate³ | | | | | | | | |
| Polyglyceryl-6 | 6 | 6 | 6.6 | 6.6 | 6 | 6 | 6 | 6 |
| Distearate (and) | | | | | | | | |
| Jojoba Esters (and) | | | | | | | | |
| Polyglyceryl-3 | | | | | | | | |
| Beeswax (and) Cetyl | | | | | | | | |
| Alcohol⁴ | | | | | | | | |
| Sorbeth-30 | | | 8 | 8 | 13 | 13 | 13 | 13 |
| Tetraoleate⁵ | | | | | | | | |
| Glyceryl laurate⁶ | | | | | | | | |
| Polyglyceryl-10 | | | | | | | | |
| Dioleate⁷ | | | | | | | | |
| Polyglyceryl-6 | 8 | 8 | | | | | | |
| Dicaprate⁸ | | | | | | | | |
| Polyglyceryl-6 | | | | | | | | |
| Myristate20 | | | | | | | | |
| Glyceryl | | | | | | | | |
| Citrate/Lactate/ | | | | | | | | |
| Linoleate/Oleate, | | | | | | | | |
| Polyglyceryl-4 | | | | | | | | |
| Cocoate, | | | | | | | | |
| Polyglyceryl-3 | | | | | | | | |
| Caprate, Glyceryl | | | | | | | | |
| Caprylate⁹ | | | | | | | | |
| Polyglyceryl-6 | | | | | | | | |
| Oleate, Potassium | | | | | | | | |
| Olivate¹⁰ | | | | | | | | |
| Structurant (wax) | | | | | | | | |
| Oryza Sativa (Rice) | | | | | | | 0.1 | 0.2 |
| Bran Wax¹¹ | | | | | | | | |
| Behenyl Behenate¹² | | | | | | | | |
| Stearyl Behenate¹³ | 3.3 | 3.3 | | | 3 | 3 | 3 | 3 |
| Tribehenin¹⁴ | 7.7 | 7.7 | 7.7 | 7.7 | 7 | 7 | 7 | 7 |
| Shea Butter¹⁵ | 2.2 | | | | | | | |
| Hydrogenated | | | 3.3 | 6.6 | | | | |
| Rapeseed oil¹⁶ | | | | | | | | |
| Hydrogenated | | | | | | | | |
| Castor Oil¹⁷ | | | | | | | | |
| C18-36 Acid | 1.1 | 1.1 | 1.1 | 1.1 | 1 | 1 | 1 | 1 |
| Triglyceride¹⁸ | | | | | | | | |
| Behenyl alcohol | | 0.5 | | | 0.5 | 0.5 | 0.5 | 0.5 |
| Tapioca starch¹⁹ | | | | | | | | |
| Botanical Extracts & | 0.56 | 0.56 | 0.56 | 0.56 | 0.56 | 0.56 | 0.56 | 0.56 |
| Essential Oils | | | | | | | | |
| Phenoxyethanol | | | | | 0.5 | | | |
| Phenylpropanol | | | | | | 0.5 | 0.5 | 0.5 |
| Stability | | | | | | | | |
| Observation | | | | | | | | |
| (weeping of oil) | | | | | | | | |
| Ease of Use | No separation | | | | | | | |
| Observation | | | | | | | | |
| (scooping & | | | | | | | | |
| spreading of balm) | | | | | | | | |
| Hardness Data (gf) | Appropriate hardness and smooth texture | | | | | | | |
| Probe Depth in | | | | | | | | |
| Sample (mm) | | | | | | | | |
| 1.0 | 197 | 150 | 73 | 70 | 58 | 56 | 75 | 75 |
| 2.0 | 277 | 270 | 118 | 121 | 76 | 74 | 101 | 92 |
| 4.0 | 292 | 302 | 154 | 178 | 105 | 94 | 137 | 133 |
| 7.0 | 426 | 328 | 194 | 209 | 120 | 110 | 146 | 189 |
| 9.5 | 357 | 453 | 198 | 219 | 107 | 108 | 183 | 162 |
| Average Hardness | 309.8 | 300.8 | 147.5 | 159.3 | 93.2 | 88.4 | 128.6 | 130.2 |
| Skin:Bulk Hardness | 0.82 | 0.67 | 0.78 | 0.81 | 0.97 | 0.87 | 0.75 | 0.82 |
| Ratio | | | | | | | | |
| Oil Binding | 92.9 | 92.6 | 70.8 | 68.6 | 66.7 | 64.2 | | |
| Capacity (%) | | | | | | | | |

| | |
|---|---|
| Overall Conclusion | Preferred |

### Suppliers for Table 1 to Table 8:

1. EMALEX GWIS-320EX, liquid (Nihon)
2. EMALEX PEIS 10 EX, paste (Nihon)
3. Span™ 83 (Croda®)
4. Emulium® Mellifera MB (Gattefossé®). Polyglyceryl-6 distearate is an emulsifier, jojoba esters, polyglyceryl-3 beeswax and cetyl alcohol are wax structurants.
5. Barcleanse STO (Barnet)
6. Ultrapure GML (Ultra Chemical)
7. Salacos® PG-218T (Ikeda Corp. of America)
8. SUNSOFT Q-102H-C(MB) (Iwase Cosfa)
9. IMWITOR® liteMULS RSPO (EES Cosmetics Solutions)
10. OLEAMULS® LOW (DKSH)
11. Naturebead® R20 (Micropowders)
12. BioMADE™ BB (ALZO International, Inc.)
13. Dermowax™ SB, prilled (ALZO International, Inc.)
14. Syncrowax™ HRC-PA-(MH) (Croda®)
15. Lipex Shea (AAK)
16. SustOleo™ TSB (Inolex®)
17. Castorwax™ MP-80 (Vertellus LLC)
18. Syncrowax™ HGLC-PA-(MH) (Croda®)
19. Tapioca Pure (Nouryon)
20. SUNSOFT Q-14Y-C(Iwase Cosfa)

### Combinations:

A. A cleansing balm composition comprising:
   a. greater than 50%, more preferably greater than 55%, and even more preferably greater than 60% of an emollient comprising a melting point below 40° C., preferably below 35° C., and more preferably below 30°; wherein the emollient is natural or naturally derived;
   b. an emulsifier;
   c. from about 8% to about 28%, preferably from about 10% to about 21%, and more preferably from about 11% to about 17% of a combination of wax structurants comprising:
      i. one or more first ester waxes comprising a melting point of at least 55° C., preferably at least about 60° C.;
      ii. one or more second ester waxes comprising a melting point of at least 45° C.; and
      iii. optionally, from about 0.2% to about 3%, preferably from about 0.3% to about 2%, and more preferably about 0.5% to about 1.5%, of one or more fatty alcohols; wherein the fatty alcohols are solid at 25° C.;
         wherein the cleansing balm is anhydrous; and
         wherein the cleansing balm composition is formulated without synthetic waxes.
B. The cleansing balm composition according to Paragraph A, wherein the emollients comprise one or more of the following: alkanes, esters, triglycerides, or a combination thereof.
C. The cleansing balm composition according to Paragraphs A-B, wherein the emulsifier comprises a non-ionic emulsifier.
D. The cleansing balm composition according to Paragraphs A-C, wherein the emulsifier comprises a non-ethoxylated emulsifier.
E. The cleansing balm composition according to Paragraphs A-D, wherein the one or more first ester waxes comprise a melting point of from 55° C. to about 70° C.
F. The cleansing balm composition according to Paragraphs A-E, comprising from about 5% to about 18%, preferably from about 8% to about 15%, and more preferably from about 10% to about 13% of the one or more first ester waxes.
G. The cleansing balm composition according to Paragraphs A-F, wherein the one or more first ester waxes comprise greater than 50%, preferably from about 55% to about 98%, more preferably from about 65% to about 97%, by weight of the combination of wax structurants.
H. The cleansing balm composition according to Paragraphs A-G, wherein the one or more first ester wax comprising stearyl behenate, tribehenin, C18-36 acid triglycerides, hydrogenated rapeseed oil, or combinations thereof.
I. The cleansing balm composition according to Paragraphs A-H, comprising from about 1% to about 12%, preferably from about 2% to about 10%, and more preferably from about 2% to about 6%, of the one or more second ester waxes.
J. The cleansing balm composition according to Paragraphs A-I, wherein the one or more second ester waxes comprise jojoba esters, trilaurin, shea butter, beeswax, polyglyceryl-3 beeswax, or a combination thereof.
K. The cleansing balm composition according to Paragraphs A-J, further comprising a third wax structurant comprising a melting point of at least 70° C.
L. The cleansing balm composition according to Paragraph K, wherein the melting point of the third wax structurant is higher than the melting point of the first ester waxes and the second ester waxes.
M. The cleansing balm composition according to Paragraphs A, wherein the composition comprises one or more fatty alcohols comprising monohydric alcohols comprising 8-22 carbon atoms.
N. The cleansing balm of according to Paragraph M, wherein the fatty alcohol is a combination of cetyl and/or behenyl alcohol.
O. The cleansing balm according to Paragraphs A-N, wherein the cleansing balm comprises a skin to bulk hardness ratio greater than 0.60 and less than 2.0, preferably from about 0.80 to about 1.5, and alternatively from about 0.80 to about 1.25, as measured according to the Penetration Method.
P. The cleansing balm according to Paragraphs A-O, wherein the cleansing balm comprises a skin to bulk hardness ratio greater than 1.0, preferably greater than 1.1, and more preferably greater than 1.2, as measured according to the Penetration Method.
Q. The cleansing balm according to Paragraphs A-P, wherein the composition comprises an average hardness of greater than 50 gf and less than 350 gf, preferably from about 50 gf to about 300 gf, more preferably from about 50 gf to about 250 gf, and even more preferably from about 50 gf to about 200 gf, as measured by the Penetration Method described herein.
R. The cleansing balm according to Paragraphs A-Q, wherein the composition comprises a % oil binding capacity of greater than 65%, preferably greater than 70%, and more preferably greater than 75%, and even more preferably greater than 80%, as measured according to the Oil Binding Capacity Test Method.
S. The cleansing balm according to Paragraphs A-R, wherein the cleansing balm is physically stable.
T. The cleansing balm according to Paragraphs A-S, wherein the composition is formulated without silicone oils, preferably the composition is free of silicone oils.
U. The cleansing balm according to Paragraphs A-T, wherein the composition is formulated without polyethylene and other petroleum derived waxes, synthetic wax, paraffins, and isoparaffins, preferably the composition is formulated without polyethylene and other petroleum derived waxes, synthetic wax, paraffins, and isoparaffins.
V. The cleansing balm according to Paragraphs A-U, wherein the one or more first ester waxes comprise stearyl behenate, C18-36 triglyceride, tribehenin, or a combination thereof.
W. The cleansing balm of according to Paragraphs A-V, wherein the one or more second ester waxes comprise jojoba esters, polyglyceryl-3 beeswax, shea butter, or a combination thereof.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A soft solid cleansing balm composition comprising:
a. greater than 50% of an emollient comprising a melting point below 40° C.; wherein the emollient is natural or naturally derived;
b. an emulsifier;
c. from about 8% to about 28% of a combination of wax structurants comprising:
i. one or more first ester waxes comprising a melting point of at least 55° C.;
ii. one or more second ester waxes comprising a melting point of at least 45° C.; and
iii. optionally, from about 0.2% to about 3% of one or more fatty alcohols;
wherein the fatty alcohols are solid at 25° C.;
wherein the cleansing balm is anhydrous; and
wherein the cleansing balm composition is formulated without synthetic waxes.

2. The cleansing balm composition of claim 1, wherein the emollients comprise alkanes, esters, triglycerides, or a combination thereof.

3. The cleansing balm composition of claim 1, wherein the emulsifier comprises a non-ionic emulsifier.

4. The cleansing balm composition of claim 3, wherein the emulsifier comprises a non-ethoxylated emulsifier.

5. The cleansing balm composition of claim 1, wherein the wax structurants comprise greater than 50%, by weight of the wax structurants, of the one or more first ester waxes.

6. The cleansing balm composition of claim 5, wherein the one or more first ester wax is chosen from stearyl behenate, tribehenin, C18-36 acid triglycerides, hydrogenated rapeseed oil, or mixtures thereof.

7. The cleansing balm composition of claim 1, comprising from about 1% to about 12% of the one or more second ester waxes.

8. The cleansing balm composition of claim 1, wherein the one or more second ester waxes is chosen from jojoba esters, trilaurin, shea butter, beeswax, polyglyceryl-3 beeswax, or mixtures thereof.

9. The cleansing balm composition of claim 1, further comprising a third wax structurant comprising a melting point of at least 70° C. and wherein the melting point of the third wax structurant is higher than the melting point of the first ester waxes and the second ester waxes.

10. The cleansing balm composition of claim 1, wherein the composition comprises the fatty alcohols and the fatty alcohols comprise monohydric alcohols comprising 8-22 carbon atoms.

11. The cleansing balm of claim 10, wherein the fatty alcohols comprise cetyl alcohol and behenyl alcohol.

12. The cleansing balm of claim 1, wherein the cleansing balm comprises a skin to bulk hardness ratio greater than 0.60 and less than 2.0, as measured according to the Penetration Method.

13. The cleansing balm of claim 1, wherein the composition comprises an average hardness of greater than 50 gf and less than 350 gf, as measured by the Penetration Method described herein.

14. The cleansing balm of claim 1, wherein the composition comprises a % oil binding capacity of greater than 65%, as measured according to the Oil Binding Capacity Test Method.

15. The cleansing balm of claim 1, preferably greater than greater than 75%, as measured according to the Oil Binding Capacity Test Method.

16. The cleansing balm of claim 1, wherein the cleansing balm is physically stable.

17. The cleansing balm of claim 1, wherein the composition is formulated without polyethylene and other petroleum derived waxes, synthetic wax, paraffins, and isoparaffins.

18. A soft solid cleansing balm composition comprising:
a. a natural or naturally derived emollient; wherein the emollient is liquid at 25° C.;
b. an emulsifier;
c. a naturally-derived wax blend comprising at least two ester waxes comprising a melting point of at least 45° C. and optionally, one or more fatty alcohols; wherein the fatty alcohols are solid at 25° C.;
wherein the cleansing balm is anhydrous;
wherein the cleansing balm composition is formulated without polyethylene;
wherein the composition comprises an average hardness of greater than 50 gf and less than 350 gf;
wherein the cleansing balm comprises a skin to bulk hardness ratio greater than 0.60 and less than 2.0; and
wherein the composition comprises an oil binding capacity percent of at least 65%.

19. A cleansing balm composition comprising:
a. a natural or naturally derived emollient; wherein the emollient is liquid at 25° C.;
b. a non-ionic emulsifier;
c. a naturally-derived wax blend comprising:
i. a C18-36 triglyceride;
ii. tribehenin;
iii. one or more ester waxes comprising jojoba esters, trilaurin, shea butter, beeswax, polyglyceryl-3 beeswax, or a combination thereof;
wherein the cleansing balm is anhydrous;
wherein the cleansing balm composition is formulated without polyethylene and silicone oils.

20. The cleansing composition of claim 19, wherein the wax structurants further comprise cetyl alcohol and behenyl alcohol.
